(19) United States
(12) Patent Application Publication
(10) Pub. No.: US 2024/0016428 A1
(43) Pub. Date: Jan. 18, 2024

(54) Integrated Catheter System with Near Patient Access Port and Visual Indicators

(71) Applicant: Becton, Dickinson and Company, Franklin Lakes, NJ (US)

(72) Inventor: Jonathan Karl Burkholz, Salt Lake City, UT (US)

(21) Appl. No.: 18/221,660

(22) Filed: Jul. 13, 2023

Related U.S. Application Data
(60) Provisional application No. 63/389,173, filed on Jul. 14, 2022.

(51) Int. Cl. A61B 5/15

(52) U.S. Cl. CPC A61B 5/150992; A61M 2039/1077

(57) ABSTRACT

An integrated catheter system includes a catheter adapter, a needle-free connector including a first port, a second port positioned opposite the first port, and a side port positioned between the first port and the second port, intermediate tubing extending between the inlet of the catheter adapter and the first port of the needle-free connector, extension tubing extending from the side port of the needle-free connector, first indicia provided on a first portion or a second portion of the needle-free connector, and second indicia provided on the first portion or the second portion of the needle-free connector. The first indicia including a first color and the second indicia including at least one of a maximum pressure rating marking, a flow rate capability marking, and a catheter length marking.

### CROSS-REFERENCE TO RELATED APPLICATION

The present application claims priority to U.S. Provisional Patent Application No. 63/389,173, entitled “Integrated Catheter System with Near Patient Access Port and Visual Indicators” filed Jul. 14, 2022, the entire disclosure of which is hereby incorporated by reference in its entirety.

### BACKGROUND OF THE INVENTION

### Field of the Invention

The present disclosure relates to an integrated catheter system with a near patient access port and visual indicators.

### Description of Related Art

Catheters are commonly used for a variety of infusion therapies. For example, catheters may be used for infusing fluids, such as normal saline solution, various medicaments, total parenteral nutrition, and high pressure injection of contrast media into a patient. Catheters may also be used for withdrawing blood from the patient.

A common type of catheter is an over-the-needle peripheral intravenous (“IV”) catheter (“PIVC”). The over-the-needle catheter may be mounted over an introducer needle having a sharp distal tip. The catheter and the introducer needle may be assembled so that the distal tip of the introducer needle extends beyond the distal tip of the catheter, with the bevel of the needle facing up away from a skin surface of the patient. The catheter and introducer needle are generally inserted at a shallow angle through the skin into the vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when an indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at a time of catheter placement but are much less frequently used for acquiring a blood sample during the catheter dwell period.

Accordingly, blood draw devices have been developed to collect blood samples through an existing PIVC. Blood draw devices attach to the PIVC and include a flexible flow tube that is advanced through the PIVC, beyond the catheter tip, and into a vessel to collect a blood sample. After blood collection, the blood draw device is removed from the PIVC and discarded. One example of a blood draw device is shown and described in U.S. Pat. No. 11,090,461, which is hereby incorporated by reference in its entirety.

### SUMMARY OF THE INVENTION

In one aspect or embodiment, an integrated catheter system includes a catheter adapter having a catheter and an inlet, with the catheter configured to be inserted into a patient's vasculature, and a needle-free connector comprising a first port, a second port positioned opposite the first port, and a side port positioned between the first port and the second port, with the second port having a valve member and the needle-free connector including a first portion and a second portion. The system further includes intermediate tubing extending between the inlet of the catheter adapter and the first port of the needle-free connector, extension tubing extending from the side port of the needle-free connector, first indicia provided on the first portion or the second portion of the needle-free connector, the first indicia including a first color, and second indicia provided on the first portion or the second portion of the needle-free connector, with the second indicia including at least one a maximum pressure rating marking, a flow rate capability marking, and a catheter length marking.

The first portion of the needle-free connector may include the first port and the side port, where the second portion of the needle-free connector includes the second port. The first indicia may be provided on the first portion of the needle-free connector. The first color of the first indicia may be indicative of high pressure injection capability. The second indicia may include the catheter length marking provided on the first portion of the needle-free connector. The second indicia may include the maximum pressure rating marking and the flow rate capability marking provided on the second portion of the needle-free connector.

The first indicia may be provided on the second portion of the needle-free connector, where the first color of the first indicia is indicative of high pressure injection capability.

The first indicia may include a second color different than the first color, where the first color of the first indicia is provided on the second portion of the needle-free connector, and where the second color of the first indicia is provided on the first portion of the needle-free connector. The first color of the first indicia may be indicative of high pressure injection capability, and the second color of the second indicia may be indicative of blood draw capability. The second indicia may include the catheter length marking provided on the first portion of the needle-free connector and the flow rate capability marking provided on the second portion of the needle-free connector.

The first indicia may be provided on the first portion of the needle-free connector, where the first color of the first indicia is indicative of blood draw capability. The first indicia may be provided on the second portion of the needle-free connector, where the first color of the first indicia is indicative of blood draw capability.

The system may include third indicia provided on the intermediate tubing, where the third indicia includes at least one of a maximum pressure rating marking, a flow rate capability marking, and a catheter length marking. A body of the needle-free connector may be opaque or transparent. The catheter may include a fenestrated tip.

The needle-free connector may include a body defining a longitudinal axis extending between the first port and the second port, where the side port extends from the body at an angle of 30-150 degrees relative to the longitudinal axis of the body. The side port may be in fluid communication with the body of the needle-free connector via an inlet, where the inlet is offset from the longitudinal axis of the body of the needle-free connector.

In one aspect or embodiment, an integrated catheter system includes a catheter adapter having a catheter and an inlet, with the catheter configured to be inserted into a patient's vasculature, a needle-free connector including a first port, a second port positioned opposite the first port, and a side port positioned between the first port and the second port, with the second port including a valve member and the needle-free connector including a first portion and a second portion, intermediate tubing extending between the inlet of the catheter adapter and the first port of the needle-free connector, extension tubing extending from the side port of the needle-free connector, and first indicia provided on the first portion or the second portion of the needle-free connector. The first indicia including at least one of a first color, a

The first indicia may be the first color, where the first color is provided on the first portion and the second portion of the needle-free connector. The catheter may include a fenestrated tip.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an integrated catheter system according to one aspect or embodiment of the present application;

FIG. 2 is a front view of a near patient access port according to one aspect or embodiment of the present application;

FIG. 3 is a cross-sectional view of the near patient access port of FIG. 2;

FIG. 4 is a side view of the near patient access port of FIG. 2;

FIG. 5 is a perspective view of an integrated catheter system according to a further aspect or embodiment of the present application;

FIG. 6 is a perspective view of an integrated catheter system according to a further aspect or embodiment of the present application;

FIG. 7 is a perspective view of an integrated catheter system according to a further aspect or embodiment of the present application;

FIG. 8 is a perspective view of an integrated catheter system according to a further aspect or embodiment of the present application;

FIG. 9 is a perspective view of an integrated catheter system according to a further aspect or embodiment of the present application;

FIG. 10 is a perspective view of an integrated catheter system according to a further aspect or embodiment of the present application;

FIG. 11 is a perspective view of an integrated catheter system according to a further aspect or embodiment of the present application;

FIG. 12 is a perspective view of an integrated catheter system according to a further aspect or embodiment of the present application;

FIG. 13 is a perspective view of an integrated catheter system according to a further aspect or embodiment of the present application;

FIG. 14 is a perspective view of an integrated catheter system according to a further aspect or embodiment of the present application; and

FIG. 15 is a perspective view of an integrated catheter system according to a further aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

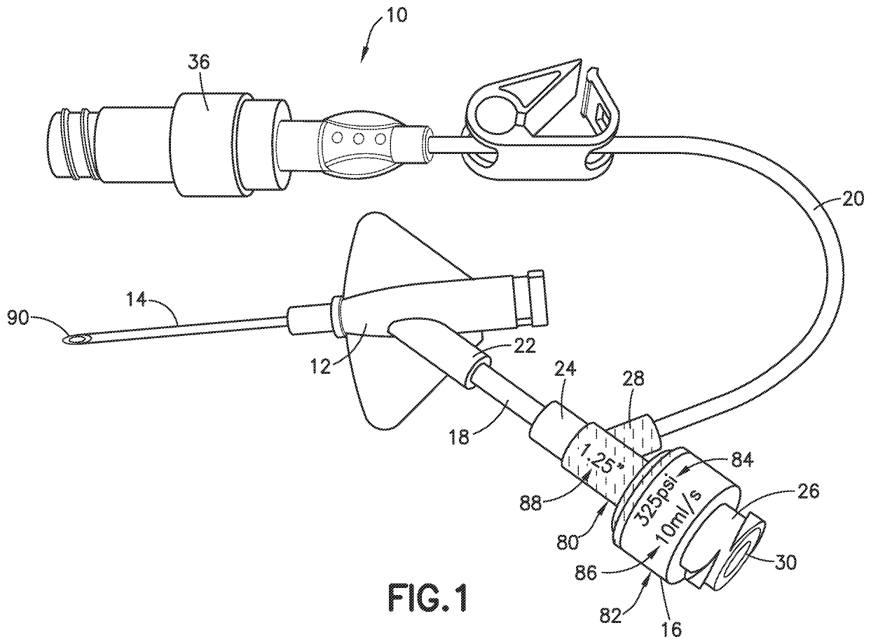

Referring to FIG. 1, an integrated catheter system 10 includes a catheter adapter 12 having a catheter 14 configured to be inserted into a patient's vasculature, a needle-free connector 16, intermediate tubing 18, and extension tubing 20. The catheter adapter 12 includes an inlet 22. The needle-free connector 16 includes a first port 24, a second port 26 positioned opposite the first port 24, and a side port 28 positioned between the first port 24 and the second port 26. The second port 26 includes a valve member 30. The intermediate tubing 18 extends between the inlet 22 of the catheter adapter 12 and the first port 24 of the needle-free connector 16. The extension tubing 20 extends from the side port 28 of the needle-free connector 16. The intermediate tubing 18 is configured to provide flexibility when inserting and dressing the catheter 14 and also when manipulating the needle-free connector 16 for flushing, blood draw, and/or other procedures without disturbing the catheter insertion site. The proximity of the needle-free connector 16 to the catheter adapter 12 is configured to enable a blood draw device to protrude a sufficient distance beyond an end of the catheter 14. The extension tubing 20 is configured to be utilized for the introducing of intravenous fluid, medication, etc.

In one aspect or embodiment, the intermediate tubing 18 has a length of 6-8 mm, although other suitable lengths may be utilized. A longer length of the intermediate tubing 18 improves insertion ergonomics, flexibility of access and securement, and ease of dressing. However, increasing the length of the intermediate tubing 18 decreases the distance the blood draw device will be able to protrude from the catheter 14 unless the blood draw device allows for a longer tubing. In one aspect or embodiment, the intermediate tubing 18 has a length that is shorter than a length of the extension tubing 20. The intermediate tubing 18 may have a length configured to allow a probe of the blood draw device to extend a predetermined length beyond a tip of the catheter 14.

Referring to FIG. 1, in one aspect or embodiment, the integrated catheter system 10 includes a needle hub assembly (not shown) and a medical component 36, such as a luer port connector. The needle hub assembly is assembled with the catheter adapter 12 by inserting a needle (not shown) into a lumen of the catheter 14. In one aspect or embodiment, the needle hub assembly includes a needle shield configured to secure a tip of the needle within the needle shield after use. The needle shield may be activated passively. In one aspect or embodiment, the catheter adapter 12 includes one or more wings, as shown, configured to engage a skin surface of a patient. In another aspect or embodiment, the catheter adapter 12 does not include wings.

When a blood draw device (not shown) is connected to the second port 26 of the needle-free connector 16, a flow tube of the blood draw device can be extended through the needle-free connector 16, the intermediate tubing 18, the inlet 22 of the catheter adapter 12, and the catheter 14. The blood draw device may be the PIVO™ blood draw device commercially available from Becton, Dickinson and Company. In one aspect or embodiment, the blood draw device is the same or similar to the blood draw device shown in U.S. Pat. No. 11,090,461, which is hereby incorporated by reference in its entirety. In one aspect or embodiment, the blood draw device may be any device that advances tubing, a probe, a guidewire, an instrument, and/or sensor into the fluid path of the integrated catheter system 10 or beyond the tip of the catheter 14.

The system 10 includes the needle-free connector 16 having optimized flush-ability features, an optimized line draw device or probe guiding fluid path, a remote luer connection to reduce side manipulation, a catheter stabilization platform, and/or a direct probe access fluid path to allow a probe to be advanced into the catheter fluid path and beyond the tip of the catheter.

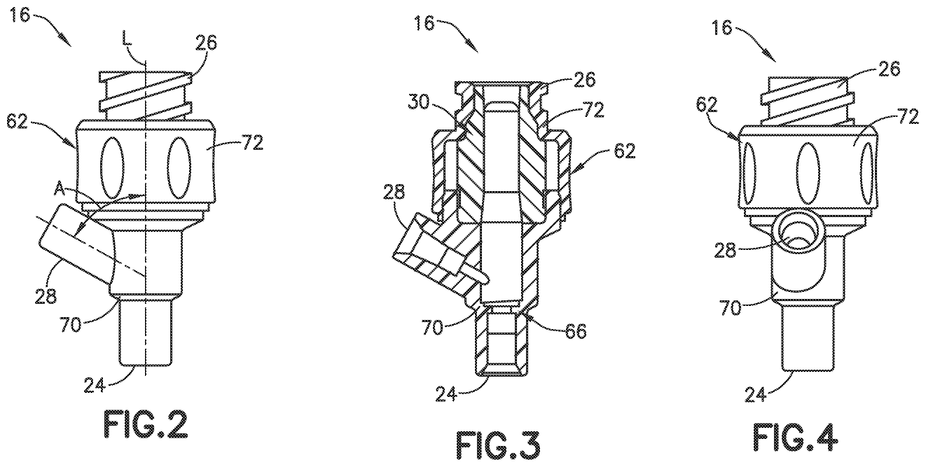

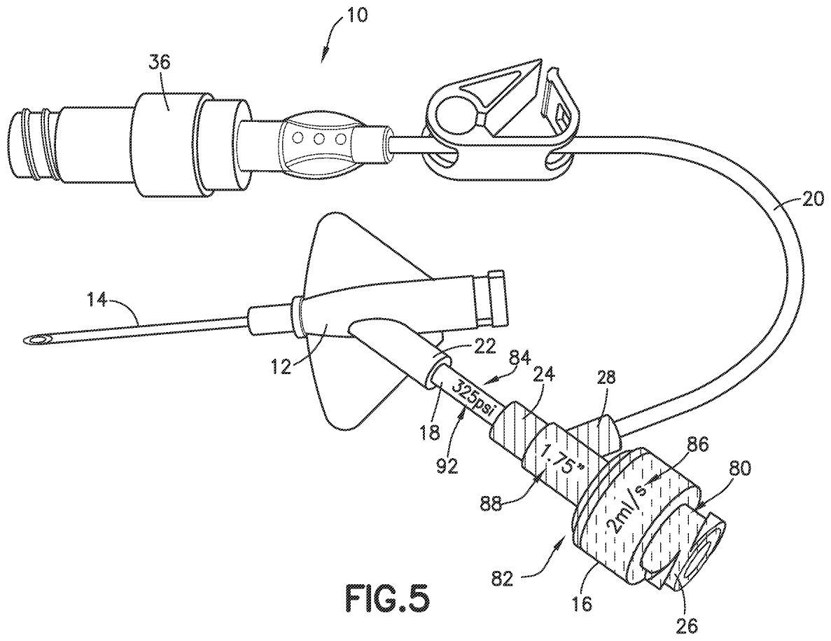

Referring to FIGS. 2-4, in one aspect or embodiment, the needle-free connector 16 includes a body 62 defining a flow path extending between the first port 24 and the second port 26. The side port 28 is offset from the center of the flow path. The offset of the side port 28 is configured to cause fluid entering the body 62 via the side port 28 to enter along an interior surface of the body 62 and cause a vortex within the body 62 to aid flushing of the needle-free connector 16. In one aspect or embodiment, the body 62 of the needle-free connector 16 further includes internal structure 66 configured to create a vortex when fluid enters the needle-free connector 16 via the side port 28. In one aspect or embodiment, the offset and vortex creating-feature is the same or similar to the flushing features shown and described in U.S. Patent Application Publication No. 2021/0220548, which is hereby incorporated by reference in its entirety.

Referring again to FIGS. 2-4, in one aspect or embodiment, the body 62 of the needle-free connector 16 defines a longitudinal axis L extending between the first port 24 and the second port 26, with the side port 28 extending from the body 62 at an angle A of 30-150 degrees relative to the longitudinal axis L of the body 62. In one aspect or embodiment, the side port 28 extends from the body 62 at an angle of 60 degrees relative to the longitudinal axis L of the body 62. The side port 28 extends at the angle A toward the second port 26, although other suitable arrangements may be utilized. The body 62 of the needle-free connector 16 includes a first portion 70 and a second portion 72 connected to the first portion 70. In one aspect or embodiment, the first portion 70 of the body 62 is fixedly secured to the second portion 72 of the body 62. In some aspects or embodiments, the body of the needle-free connector is opaque or transparent.

Referring to FIGS. 1-15, in one aspect or embodiment, the system 10 includes first indicia 80 provided on the first portion 70 or the second portion 72 of the needle-free connector 16 and second indicia 82 provided on the first portion 70 or the second portion 72 of the needle-free connector 16. The first indicia 80 is a first color and the second indicia 82 includes at least one of a maximum pressure rating marking 84, a flow rate capability marking 86, and a catheter length marking 88. The system 10 is configured to be used for a high-pressure injection procedure, with the first and second indicia 80, 82 configured to provide a healthcare worker with the capabilities of the system 10 as well as indications as to which port to use for high-pressure injection procedures and/or blood draw or other procedures. In some aspects or embodiments, the first portion 70 of the needle-free connector 16 includes the first port 24 and the side port 28, and the second portion 72 of the needle-free connector 16 includes the second port 26, although the second portion 72 could also include the side port 28. In some aspects or embodiments, the catheter 14 includes a fenestrated tip 90.

Referring to FIG. 1, in one aspect or embodiment, the first indicia 80 is provided on the first portion 70 of the needle-free connector 16. The first color of the first indicia 80 may be indicative of high pressure injection capability. In one aspect or embodiment, the first color is purple, although other distinct or contrasting colors may be utilized. As shown in FIG. 1, the second indicia 82 includes the catheter length marking 88 provided on the first portion 70 of the needle-free connector 16 and the maximum pressure rating marking 84 and the flow rate capability marking 86 provided on the second portion 72 of the needle-free connector 16.

Referring to FIG. 5, in one aspect or embodiment, the first indicia 80 includes a second color different than the first color. The first color of the first indicia 80 is provided on the second portion 72 of the needle-free connector 16, and the second color of the first indicia 80 is provided on the first portion 70 of the needle-free connector 16. The second color of the second indicia 82 is indicative of blood draw capability. As shown in FIG. 5, the first color is purple and the second color is red, although other distinct or contrasting colors may be utilized. The second indicia 82 includes the catheter length marking 88 provided on the first portion 70 of the needle-free connector 16 and the flow rate capability marking 86 provided on the second portion 72 of the needle-free connector 16. The system 10 may include third indicia 92 provided on the intermediate tubing 18, with the third indicia 92 including at least one of the maximum pressure rating marking 84, the flow rate capability marking 86, and the catheter length marking 88. As shown in FIG. 5, the third indicia 92 is the maximum pressure rating marking 84.

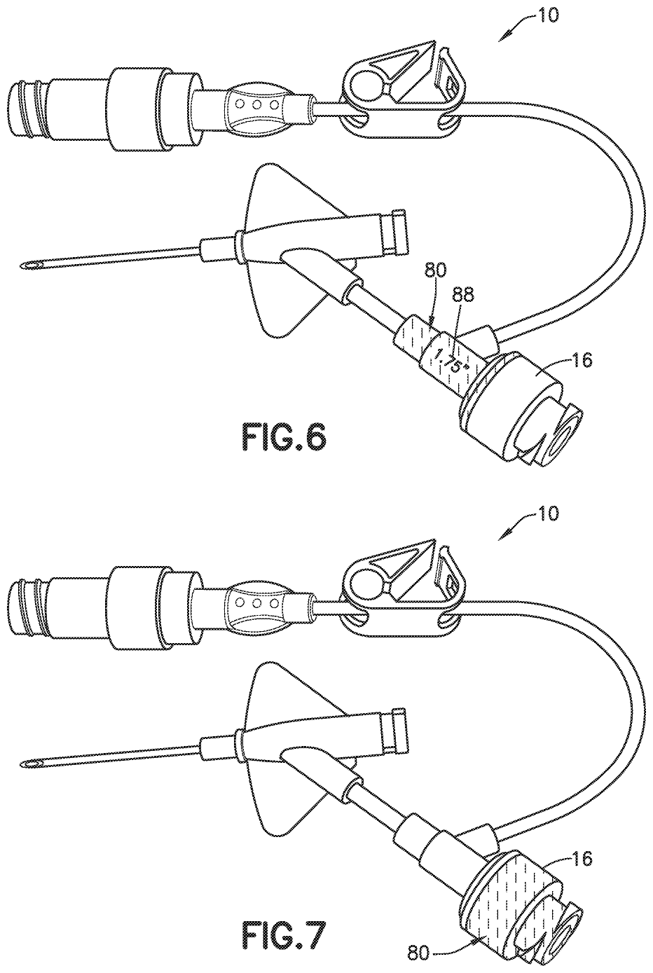

Referring to FIG. 6, in one aspect or embodiment, the first indicia 80 is provided on the first portion 70 of the needle-free connector 16 and the second indicia 82 is the catheter length marking 88 provided on the first portion 70 of the needle-free connector 16.

Referring to FIG. 7, in one aspect or embodiment, the first indicia 80 is provided on the second portion 72 of the needle-free connector 16, with the first color of the first indicia 80 is indicative of high pressure injection capability.

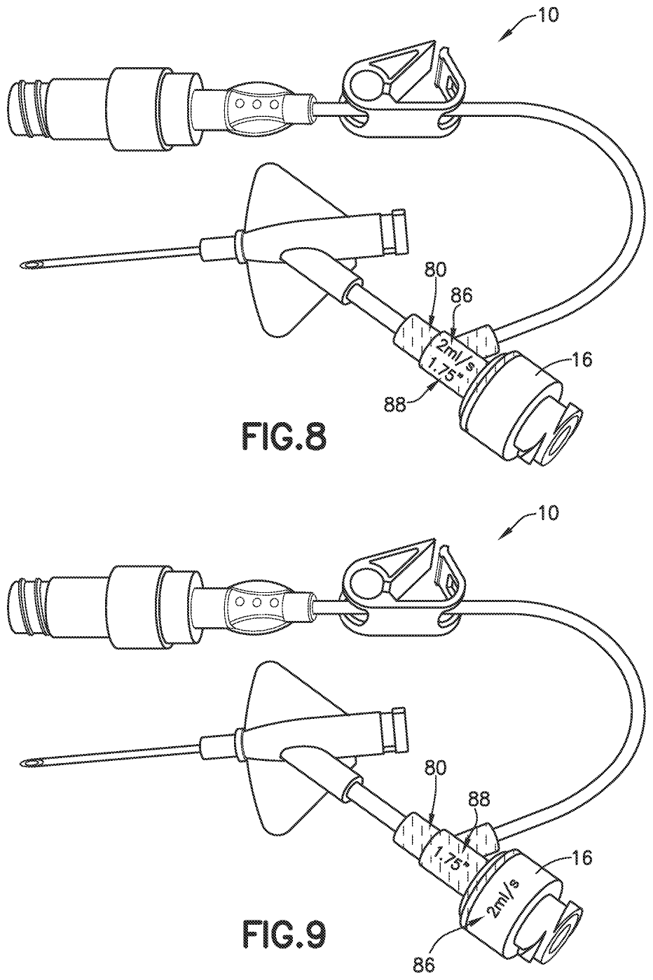

Referring to FIG. 8, in one aspect or embodiment, the first indicia 80 is provided on the first portion 70 of the needle-free connector 16 and the second indicia 82 is the catheter length marking 88 and the flow rate capability marking 86 provided on the first portion 70 of the needle-free connector 16.

Referring to FIG. 9, in one aspect or embodiment, the first indicia 80 is provided on the first portion 70 of the needle-free connector 16 and the second indicia 82 is the catheter length marking 88 provided on the first portion 70 of the needle-free connector 16 and the flow rate capability marking 86 provided on the second portion 72 of the needle-free connector 16.

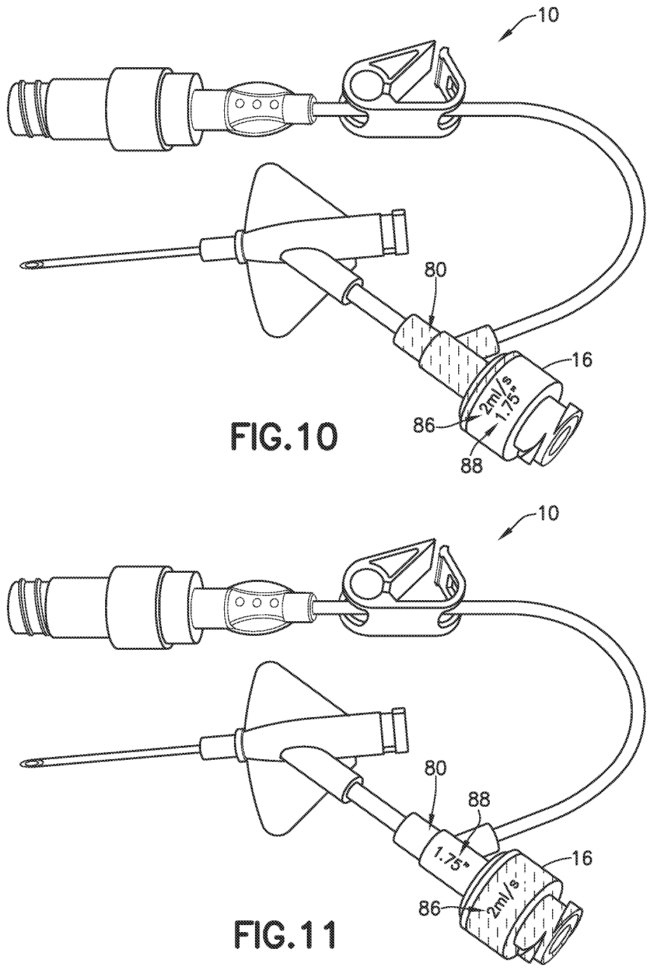

Referring to FIG. 10, in one aspect or embodiment, the first indicia 80 is provided on the first portion 70 of the needle-free connector 16 and the second indicia 82 is the catheter length marking 88 and the flow rate capability marking 86 provided on the second portion 72 of the needle-free connector 16.

Referring to FIG. 11, in one aspect or embodiment, the first indicia 80 is provided on the second portion 72 of the needle-free connector 16 and the second indicia 82 is the catheter length marking 88 provided on the first portion 70 of the needle-free connector 16 and the flow rate capability marking 86 provided on the second portion 72 of the needle-free connector 16. In the aspects or embodiments shown in FIGS. 6-11, the first indicia 80 is the first color indicative of a high pressure injection capability, such as purple.

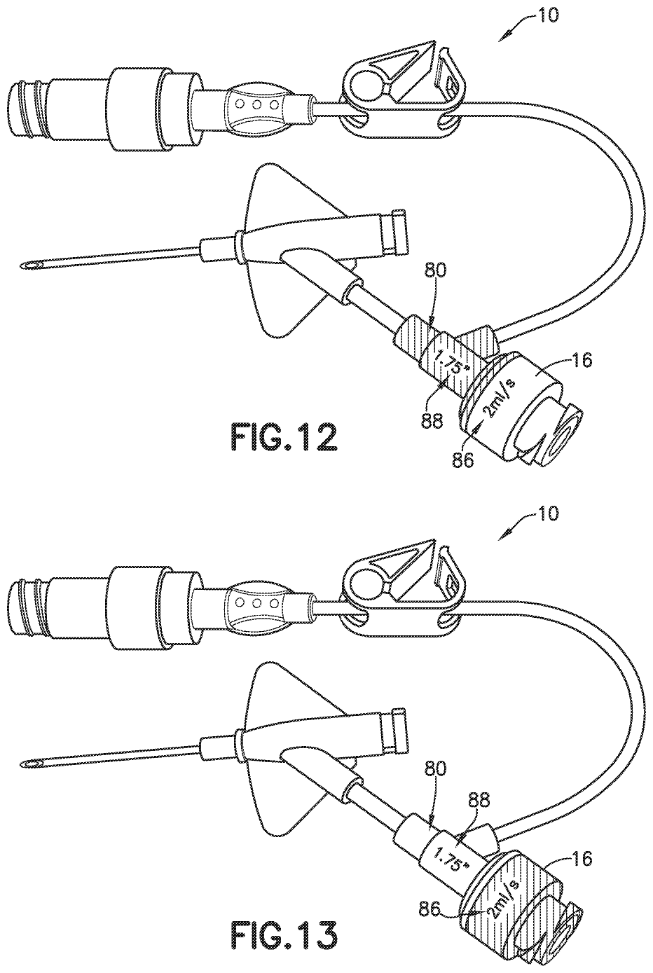

Referring to FIG. 12, in one aspect or embodiment, the first indicia 80 is provided on the first portion 70 of the needle-free connector 16 and the second indicia 82 is the catheter length marking 88 provided on the first portion 70 of the needle-free connector 16 and the flow rate capability marking 86 provided on the second portion 72 of the needle-free connector 16.

Referring to FIG. 13, in one aspect or embodiment, the first indicia 80 is provided on the second portion 72 of the needle-free connector 16 and the second indicia 82 is the catheter length marking 88 provided on the first portion 70 of the needle-free connector 16 and the flow rate capability marking 86 provided on the second portion 72 of the needle-free connector 16.

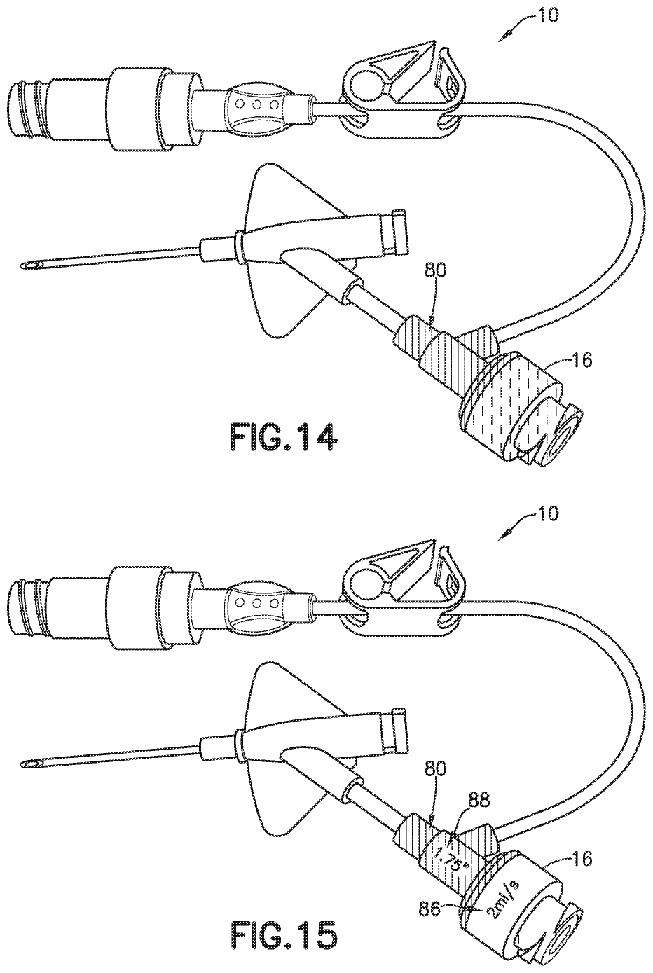

Referring to FIG. 14, in one aspect or embodiment, the first indicia 80 is the second color provided on the first portion 70 of the needle-free connector 16 and the first color provided on the second portion 72 of the needle-free connector 16. As discussed above, the first color is indicative of a high pressure injection capability, such as purple, and the second color is indicative of a blood draw capability, or for use as an arterial catheter, such as red.

Referring to FIG. 15, in one aspect or embodiment, the first indicia 80 is the second color provided on the first portion 70 of the needle-free connector 16 and the second indicia 82 is the catheter length marking 88 provided on the first portion 70 of the needle-free connector 16 and the flow rate capability marking 86 provided on the second portion 72 of the needle-free connector 16.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An integrated catheter system comprising:
a catheter adapter comprising a catheter and an inlet, the catheter configured to be inserted into a patient's vasculature;
a needle-free connector comprising a first port, a second port positioned opposite the first port, and a side port positioned between the first port and the second port, the second port comprising a valve member, the needle-free connector comprising a first portion and a second portion;
intermediate tubing extending between the inlet of the catheter adapter and the first port of the needle-free connector;
extension tubing extending from the side port of the needle-free connector;
first indicia provided on the first portion or the second portion of the needle-free connector, the first indicia comprising a first color; and
second indicia provided on the first portion or the second portion of the needle-free connector, the second indicia comprising at least one of a maximum pressure rating marking, a flow rate capability marking, and a catheter length marking.

2. The system of claim 1, wherein the first portion of the needle-free connector includes the first port and the side port, and wherein the second portion of the needle-free connector includes the second port.

3. The system of claim 2, wherein the first indicia is provided on the first portion of the needle-free connector.

4. The system of claim 3, wherein the first color of the first indicia is indicative of high pressure injection capability.

5. The system of claim 3, wherein the second indicia comprises the catheter length marking provided on the first portion of the needle-free connector.

6. The system of claim 5, wherein the second indicia comprises the maximum pressure rating marking and the flow rate capability marking provided on the second portion of the needle-free connector.

7. The system of claim 2, wherein the first indicia is provided on the second portion of the needle-free connector, and wherein the first color of the first indicia is indicative of high pressure injection capability.

8. The system of claim 2, wherein the first indicia comprises a second color different than the first color, wherein the first color of the first indicia is provided on the second portion of the needle-free connector, and wherein the second color of the first indicia is provided on the first portion of the needle-free connector.

9. The system of claim 8, wherein the first color of the first indicia is indicative of high pressure injection capability, and wherein the second color of the second indicia is indicative of blood draw capability.

10. The system of claim 9, wherein the second indicia comprises the catheter length marking provided on the first portion of the needle-free connector and the flow rate capability marking provided on the second portion of the needle-free connector.

11. The system of claim 2, wherein the first indicia is provided on the first portion of the needle-free connector, and wherein the first color of the first indicia is indicative of blood draw capability.

12. The system of claim 2, wherein the first indicia is provided on the second portion of the needle-free connector, and wherein the first color of the first indicia is indicative of blood draw capability.

13. The system of claim 1, further comprising third indicia provided on the intermediate tubing, wherein the third indicia comprises at least one of a maximum pressure rating marking, a flow rate capability marking, and a catheter length marking.

14. The system of claim 1, wherein a body of the needle-free connector is opaque or transparent.

15. The system of claim 1, wherein the catheter comprises a fenestrated tip.

16. The system of claim 1, wherein the needle-free connector comprises a body defining a longitudinal axis extending between the first port and the second port, and wherein the side port extends from the body at an angle of 30-150 degrees relative to the longitudinal axis of the body.

17. The system of claim 1, wherein the side port is in fluid communication with the body of the needle-free connector via the inlet, and wherein the inlet is offset from the longitudinal axis of the body of the needle-free connector.

18. An integrated catheter system comprising:
a catheter adapter comprising a catheter and an inlet, the catheter configured to be inserted into a patient's vasculature;
a needle-free connector comprising a first port, a second port positioned opposite the first port, and a side port positioned between the first port and the second port, the second port comprising a valve member, the needle-free connector comprising a first portion and a second portion;
intermediate tubing extending between the inlet of the catheter adapter and the first port of the needle-free connector;
extension tubing extending from the side port of the needle-free connector; and
first indicia provided on the first portion or the second portion of the needle-free connector, the first indicia comprising at least one of a first color, a maximum pressure rating marking, a flow rate capability marking, and a catheter length marking.

19. The system of claim 18, wherein the first indicia comprises the first color, and wherein the first color is provided on the first portion and the second portion of the needle-free connector.

20. The system of claim 18, wherein the catheter comprises a fenestrated tip.
